(19) United States
(12) Patent Application Publication
(10) Pub. No.: US 2015/0266836 A1
(43) Pub. Date: Sep. 24, 2015

(54) AROMATIC SULFIDE COMPOUNDS AND METHODS AND USE THEREOF

(71) Applicant: The Regents of the University of California, Oakland, CA (US)

(72) Inventors: Ivelina M. Yonova, Irvine, CA (US); Charlotte A. Osborne, Irvine, CA (US); Naomi S. Morrissette, Irvine, CA (US); Elizabeth R. Jarvo, Irvine, CA (US)

(21) Appl. No.: 14/666,088

(22) Filed: Mar. 23, 2015

Related U.S. Application Data
(60) Provisional application No. 61/969,031, filed on Mar. 21, 2014.

(51) Int. Cl. C07D 263/58; C07D 271/113

(52) U.S. Cl. CPC C07D 263/58; C07D 271/113

(57) ABSTRACT

Described herein, inter alia, are aromatic sulfide compositions and methods for treating or preventing cancer using the same.

### CROSS-REFERENCES TO RELATED APPLICATIONS

This application claims the benefit of the U.S. Provisional Patent Application No. 61/969,031, filed Mar. 21, 2014, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under P30CA062203 and F31CA177212 awarded by the National Cancer Institutes of the National Institutes of Health. The government has certain rights in the invention.

### BACKGROUND OF THE INVENTION

Cancer still remains an enormous burden on today's society. Over the past decade, the occurrence of cancer has increased due to the growth and aging of the world population, as well as an increasing prevalence of established risk factors such as smoking, overweight, physical inactivity, and changing reproductive patterns associated with urbanization and economic development. Based on GLOBOCAN estimates, about 14.1 million of new cancer cases and 8.2 million deaths occurred back in 2012 worldwide.

Despite of researches continuously searching for new drugs and treatment options to fight cancer, the survival rate of patients with some cancers still remains low. For example, lung, colon, prostate, and breast cancers continue to be the most common causes of cancer death, accounting for almost half of the total cancer deaths among men and women. More than 1 out of every 4 cancer deaths is due to lung cancer and an estimated 40,430 breast cancer deaths (40,000 women, 430 men) were expected back in 2014. Breast cancer in particular ranks second as a cause of cancer death in women (after lung cancer). Although early detection methods generally have improved for some cancers, the development of new treatment options has not advanced at the same rate. Therefore there is a great need to develop new anti-cancer compounds. Provided herein are solutions to these and other problems in the art.

### BRIEF SUMMARY OF THE INVENTION

Provided herein, inter alia, are aromatic sulfide compounds and methods of using and synthesizing the same.

In one aspect, a compound is provided having a formula:

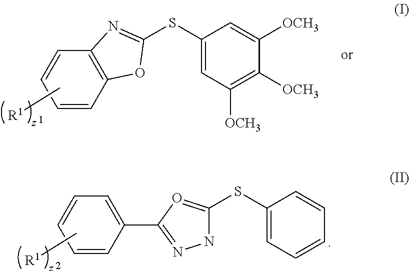

R¹ is independently a halogen, —NR²R³, —CX^{a}₃, —CHX^{a}₂, —CH₂X^{a}, —CN, —SO₂Cl, —SOₙ₁R⁴, —SOₙ₁NR²R³, —NHNR²R³, —ONR²R³, —NHC(O)NHNR²R³, —NHC(O)NR²R³, —N(O)ₘ₁, —C(O)R⁵, —C(O)OR⁵, —C(O)NR²R³, —OR⁴, —NR²SO₂R⁴, —NR²C(O)OR⁵, —NR²C(O)R⁵, —N₃, —NR²OR⁵, —OCX^{a}₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R², R³, R⁴, R⁵, R⁶, R⁷, R⁸, and R⁹ are independently hydrogen, halogen, —CX^{b}₃, —CHX^{b}₂, —CH₂X^{b}, —CN, —SO₂Cl, —SOₙ₂R⁶, —SOₙ₂NR⁷R⁸, —NHNH₂, —ONR⁷R⁸, —NHC(O)NHNH₂, —NHC(O)NR⁷R⁸, —N(O)ₘ₂, —NR⁷R⁸, —C(O)R⁹, —C(O)OR⁹, —C(O)NR⁷R⁸, —OR⁶, —NR⁷SO₂R⁶, —NR⁷C(O)OR⁹, —NR⁷C(O)R⁹, —NR⁷OR⁹, —OCX^{b}₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R³ are optionally joined to form a substituted or unsubstituted heterocycloalkyl, or substituted or unsubstituted heteroaryl; R⁷ and R⁸ are optionally joined to form a substituted or unsubstituted heterocycloalkyl or substituted or unsubstituted heteroaryl.

The symbol z1 is an integer from 0 to 4. The symbol z2 is an integer from 0 to 5. The symbols m1 and m2 are independently 1 or 2. The symbols n1 and n2 are independently an integer from 0 to 2. The symbols X^{a} and X^{b} are independently —Cl, —Br, —I, or −F.

In embodiments, R¹ is halogen, —NR²R³, —CX^{a}₃, —CN, —SO₂Cl, —SOₙ₁R⁴, —SOₙ₁NR²R³, —NHNR²R³, —ONR²R³, —NHC(O)NHNR²R³, —NHC(O)NR²R³, —N(O)ₘ₁, —C(O)R⁵, —C(O)OR⁵, —C(O)NR²R³, —OR⁴, —NR²SO₂R⁴, —NR²C(O)R⁵, —NR²C(O)OR⁵, —NR²OR⁵, —OCX^{a}₃, —N₃, R^{1a}-substituted or unsubstituted alkyl, R^{1a}-substituted or unsubstituted heteroalkyl, R^{1a}-substituted or unsubstituted cycloalkyl, R^{1a}-substituted or unsubstituted heterocycloalkyl, R^{1a}-substituted or unsubstituted aryl, or R^{1a}-substituted or unsubstituted heteroaryl.

R^{1a} is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)NH₂, —NO₂, —C(O)H, —C(O)OH, —C(O)NH₂, —OH, —NHSO₂H, —NHC(O)H, —NHC(O)OH, —NHOH, —OCF₃, oxo, —N₃, R^{1b}-substituted or unsubstituted alkyl, R^{1b}-substituted or unsubstituted heteroalkyl, R^{1b}-substituted or unsubstituted cycloalkyl, R^{1b}-substituted or unsubstituted heterocycloalkyl, R^{1b}-substituted or unsubstituted aryl, or R^{1b}-substituted or unsubstituted heteroaryl.

R^{1b} is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

In embodiments, R^{1b} is unsubsituted C₁-C₅ alkyl.

In embodiments, R², R³, R⁴, R⁵, R⁶, R⁷, R⁸ and R⁹ are independently selected from hydrogen, halogen, —CX^{b}₃, —CHX^{b}₂, —CH₂X^{b}, —CN, —SO₂Cl, —SOₙ₂R⁶, —SOₙ₂NR⁷R⁸, —NHNH₂, —ONR⁷R⁸, —NHC(O)NHNH₂, —NHC(O)NR⁷R⁸, —N(O)ₘ₂, —NR⁷R⁸, —C(O)R⁹, —C(O)OR⁹, —C(O)NR⁷R⁸, —OR⁶, —NR⁷SO₂R⁶, —NR⁷C(O)R⁹, —NR⁷C(O)OR⁹, —NR⁷OR⁹, —OCX^{b}₃, R¹⁰-substituted or unsubstituted alkyl, R¹⁰-substituted or unsubstituted heteroalkyl, R¹⁰-substituted or unsubstituted cycloalkyl, R¹⁰-substituted or unsubstituted heterocycloalkyl, R¹⁰-substituted or unsubstituted aryl, or R¹⁰-substituted and unsubstituted heteroaryl.

In embodiments, R² and R³ substituents are optionally joined to form R¹⁰-substituted or unsubstituted heterocycloalkyl, or R¹⁰-substituted or unsubstituted heteroaryl and R⁷ and R⁸ substituents are optionally joined to form R¹⁰-substituted or unsubstituted heterocycloalkyl, or R¹⁰-substituted or unsubstituted heteroaryl.

R¹⁰ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)NH₂, —NO₂, —C(O)H, —C(O)OH, —C(O)NH₂, —OH, —NHSO₂H, —NHC(O)H, —NHC(O)OH, —NHOH, —OCF₃, —N₃, oxo, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted and unsubstituted heteroaryl.

R¹¹ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)NH₂, —NO₂, —C(O)H, —C(O)OH, —C(O)NH₂, —OH, —NHSO₂H, —NHC(O)H, —NHC(O)OH, —NHOH, —OCF₃, —N₃, oxo, unsubstituted alkyl, unsubstituted heteroalkyl, unsubstituted cycloalkyl, unsubstituted heterocycloalkyl, unsubstituted aryl, or unsubstituted heteroaryl.

In embodiments, R¹¹ is unsubstituted C₁-C₅ alkyl.

In embodiments, R² and R³ substituents are joined to form R¹⁰-substituted or unsubstituted heterocycloalkyl, or R¹⁰-substituted or unsubstituted heteroaryl.

In embodiments, R² and R³ substituents are joined to form R¹⁰-substituted or unsubstituted heterocycloalkyl and R¹⁰ is unsubsituted C₁-C₅ alkyl.

In embodiments, R⁷ and R⁸ substituents are joined to form R¹⁰-substituted or unsubstituted heterocycloalkyl, or R¹⁰-substituted or unsubstituted heteroaryl.

In embodiments, R⁷ and R⁸ substituents are joined to form R¹⁰-substituted or unsubstituted heterocycloalkyl and R¹⁰ is unsubsituted C₁-C₅ alkyl.

In embodiments, R^{1b} is methyl.

In embodiments, R^{1a} is R^{1b}-unsubstituted alkyl.

In embodiments, R^{1a} is R^{1b}-unsubstituted alkyl and z1 is 2

In embodiments, R^{1a} is R^{1b}-unsubstituted alkyl and z1 is 1.

In embodiments, R^{1a} is R^{1b}-unsubstituted alkyl and z2 is 2.

In embodiments R^{1a} is R^{1b}-unsubstituted alkyl and z2 is 1.

In embodiments, a compound of formula (I) and z1 is 0.

In embodiments, a compound of formula (II) and z2 is 0.

In another aspect, a pharmaceutical composition including a pharmaceutically acceptable excipient and a compound or a pharmaceutically acceptable salt described herein is provided.

In another aspect, a method of treating cancer in a patient in need of such treatment. The method including administering to the patient an effective amount of a compound or a pharmaceutically acceptable salt described herein, to the patient.

### BRIEF DESCRIPTION OF THE DRAWINGS

FIG. 1. Evaluation of diaryl sulfides for anti-breast cancer activity. Anti-breast cancer activity of compounds at 10 μM screened against breast cancer (MCF-7) and normal breast cell lines (MCF-10A). Cell proliferation is presented as relative cell numbers after treatment.

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, B, As, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

The terms “cycloalkyl” and “heterocycloalkyl,” by themselves or in combination with other terms, mean, unless otherwise stated, non-aromatic cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be a —O— bonded to a ring heteroatom nitrogen.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)N R′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″, —CN, —NO₂, —NR′SO₂R″, —NR′C═(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C═(O)NR″NR′″R″, —CN, —NO₂, —R′, —N₃, —CH(Ph)₂, fluoro(C₁-C₄)alkoxy, and fluoro(C₁-C₄)alkyl, —NR′SO₂R″, —NR′C═(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)^{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include, oxygen (O), nitrogen (N), sulfur (S), phosphorus (P), Boron (B), Arsenic (As), and silicon (Si).

A “substituent group,” as used herein, means a group selected from the following moieties:
(A) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₅ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and/or each substituted or unsubstituted heterocycloalkyl is a substituted or unsubstituted 3 to 8 membered heterocycloalkyl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₅ cycloalkylene, and/or each substituted or unsubstituted heterocycloalkylene is a substituted or unsubstituted 3 to 8 membered heterocycloalkylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and/or each substituted or unsubstituted heterocycloalkyl is a substituted or unsubstituted 5 to 7 membered heterocycloalkyl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₅-C₇ cycloalkylene, and/or each substituted or unsubstituted heterocycloalkylene is a substituted or unsubstituted 5 to 7 membered heterocycloalkylene.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853). The methods above may be used to synthesize single molecular species.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat diseases associated with estrogen receptor activity. Certain methods described herein may treat diseases associated with estrogen receptor activity (e.g., breast cancer, lung cancer, a gynecological cancer, ovarian cancer, endometrial cancer, or prostate cancer, lymphangioleiomyomatosis (LAM)) by inhibiting estrogen receptor activity. Certain methods described herein may treat diseases associated with estrogen receptor activity by inhibiting ligand binding to estrogen receptor. Certain methods described herein may treat diseases associated with estrogen receptor activity by inducing the degradation of estrogen receptor. Certain methods described herein may treat diseases associated with estrogen receptor activity by inducing a non-active conformation of estrogen receptor. Certain methods described herein may treat diseases associated with hyperproliferation (e.g., of cells). For example, certain methods herein treat cancer. For example certain methods herein treat cancer by decreasing a symptom of cancer. Symptoms of cancer would be known or may be determined by a person of ordinary skill in the art. The term “treating” and conjugations thereof, include prevention of an injury, pathology, condition, or disease.

An “effective amount” is an amount sufficient for a compound to accomplish a stated purpose relative to the absence of the compound (e.g. achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g. hyperproliferative disease, cancer) means that the disease is caused by (in whole or in part), or a symptom of the disease is caused by (in whole or in part) the substance or substance activity or function. As used herein, what is described as being associated with a disease, if a causative agent, could be a target for treatment of the disease. For example, a disease associated with estrogen receptor activity may be treated with an agent (e.g. compound as described herein) effective for decreasing the level of estrogen receptor activity.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As defined herein, the term “inhibition”, “inhibit”, “inhibiting” and the like in reference to a protein-inhibitor (e.g. antagonist) interaction means negatively affecting (e.g. decreasing) the level of activity or function of the protein relative to the level of activity or function of the protein in the absence of the inhibitor. In some embodiments inhibition refers to reduction of a disease or symptoms of disease. Thus, inhibition may include, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein relative to the activity or function of the protein in the absence of the activator (e.g. compound described herein). Thus, activation may include, at least in part, partially or totally increasing stimulation, increasing or enabling activation, or activating, sensitizing, or up-regulating signal transduction or enzymatic activity or the amount of a protein decreased in a disease. Activation may include, at least in part, partially or totally increasing stimulation, increasing or enabling activation, or activating, sensitizing, or up-regulating signal transduction or enzymatic activity or the amount of a protein.

The term “modulator” refers to a composition that increases or decreases the level of a target molecule or the function of a target molecule. In embodiments, a modulator is an anti-cancer agent. In embodiments, a modulator is an estrogen receptor antagonist. In embodiments, a modulator is a hormone receptor antagonist. In embodiments, a modulator is an estrogen receptor inhibitor. In embodiments, a modulator is an estrogen receptor covalent modifier.

“Anti-cancer agent” or “anti-cancer drug” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent approved by the FDA or similar regulatory agency of a country other than the USA, for treating cancer. Examples of anti-cancer agents include, but are not limited to, anti-androgens (e.g., Casodex, Flutamide, MDV3100, or ARN-509), MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine), anti-metabolites (e.g., 5-azathioprine, leucovorin, capecitabine, fludarabine, gemcitabine, pemetrexed, raltitrexed, folic acid analog (e.g., methotrexate), pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib (Gleevec®), geldanamycin, 17-N-Allylamino-17-Demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, pyrrolo benzodiazepines (e.g. tomaymycin), carboplatin, CC-1065 and CC-1065 analogs including amino-CBIs, nitrogen mustards (such as chlorambucil and melphalan), dolastatin and dolastatin analogs (including auristatins: eg. monomethyl auristatin E), anthracycline antibiotics (such as doxorubicin, daunorubicin, etc.), duocarmycins and duocarmycin analogs, enediynes (such as neocarzinostatin and calicheamicins), leptomycin derivaties, maytansinoids and maytansinoid analogs (e.g. mertansine), methotrexate, mitomycin C, taxoids, vinca alkaloids (such as vinblastine and vincristine), epothilones (e.g. epothilone B), fluvestrant, camptothecin and its clinical analogs topotecan and irinotecan, SERMS (e.g., clomifene, femarelle, ormeloxifene, raloxifene, tamoxifen, toremifene, lasofoxifene, ospemifene), aromatase inhibitors (e.g., anastrozole, letrozole, exemestane, vorozole, formestane, fadrozole, aminoglutethimide, testolactone), or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with a compound, pharmaceutical composition, or method provided herein. In some embodiments, the disease is a disease having the symptom of cell proliferation. In some embodiments, the disease is a cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma. In embodiments, the disease is breast cancer. In embodiments, the disease is hormone sensitive breast cancer. In embodiments, the disease is hormone refractory (insensitive) breast cancer. In embodiments, the disease is ER positive breast cancer. In embodiments, the disease is ER negative breast cancer. In embodiments, the disease is breast cancer expressing HER-2.

As used herein, the term “cancer” refers to all types of cancer, neoplasm or malignant tumors found in mammals (e.g. humans), including leukemia, carcinomas and sarcomas. Exemplary cancers that may be treated with a compound or method provided herein include cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

The term “ER positive” breast cancer refers to a type of breast cancer that grows in response to the hormone estrogen. If the tumor has a significant number of estrogen receptors, then the cancer is considered hormone-receptor positive and likely to respond to endocrine therapies. Likewise if the breast cancer is “ER negative” alternative treatment options must be chosen.

The term “HER2-Positive” breast cancer refers to cancer cells that make too much of a protein known as HER2/neu. These breast cancers tend to be much more aggressive and fast-growing. For women with HER2-positive breast cancers, the drug Herceptin has been shown to dramatically reduce the risk of recurrence. It has now become standard treatment to give Herceptin along with adjuvant (after-surgery) chemotherapy in those with metastatic breast cancer.

The term “triple negative” breast cancer refers to cancer cells that lack estrogen and progesterone receptors and do not overexpress the HER2 protein. These cancers generally respond well to adjuvant chemotherapy. Overall, however, they have a poorer prognosis than other types of breast cancer.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation, to increase degradation of a prodrug and release of the drug, detectable agent). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

### II. Compounds

Provided herein compositions having a compound of formula

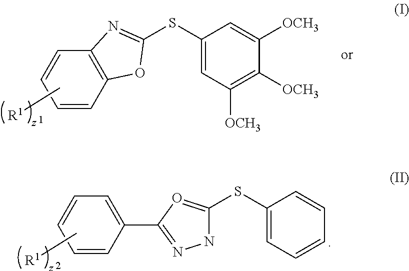

R¹ is independently a halogen, —NR²R³, —CX^{a}₃, —CHX^{a}₂, —CH₂X^{a}, —CN, —SO₂Cl, —SOₙ₁R⁴, —SOₙ₁NR²R³, —NHNR²R³, —ONR²R³, —NHC(O)NHNR²R³, —NHC(O)NR²R³, —N(O)ₘ₁, —C(O)R⁵, —C(O)OR⁵, —C(O)NR²R³, —OR⁴, —NR²SO₂R⁴, —NR²C(O)R⁵, —NR²C(O)OR⁵, —N₃, —000, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R², R³, R⁴, R⁵, R⁶, R⁷, R⁸, and R⁹ are independently hydrogen, halogen, —CX^{b}₃, —CHX^{b}₂, —CH₂X^{b}, —CN, —SO₂Cl, —SOₙ₂R⁶, —SOₙ₂NR⁷R⁸, —NHNH₂, —ONR⁷R⁸, —NHC(O)NHNH₂, —NHC(O)NR⁷R⁸, —N(O)ₘ₂, —NR⁷R⁸, —C(O)R⁹, —C(O)OR⁹, —C(O)NR⁷R⁸, —OR⁶, —NR⁷SO₂R⁶, —NR⁷C(O)R⁹, —NR⁷C(O)OR⁹, —NR⁷OR⁹, —OCX^{b}₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² and R³ are optionally joined to form a substituted or unsubstituted heterocycloalkyl, or substituted or unsubstituted heteroaryl; R⁷ and R⁸ are optionally joined to form a substituted or unsubstituted heterocycloalkyl or substituted or unsubstituted heteroaryl.

The symbol z1 is an integer from 0 to 4. The symbol z2 is an integer from 0 to 5. The symbols m1 and m2 are independently 1 or 2. The symbols n1 and n2 are independently an integer from 0 to 2. The symbols X^{a} and X^{b} are independently —Cl, —Br, —I, or —F.

In embodiments, R¹ is halogen, —NR²R³, —CX^{a}₃, —CN, —SO₂Cl, —SOₙ₁R⁴, —SOₙ₁NR²R³, —NHNR²R³, —ONR²R³, —NHC(O)NHNR²R³, —NHC(O)NR²R³, —N(O)ₘ₁, —C(O)R⁵, —C(O)OR⁵, —C(O)NR²R³, —OR⁴, —NR²SO₂R⁴, —NR²C(O)R⁵, —NR²C(O)OR⁵, —NR²OR⁵, —OCX^{a}₃, —N₃, R^{1a}-substituted or unsubstituted alkyl, R^{1a}-substituted or unsubstituted heteroalkyl, R^{1a}-substituted or unsubstituted cycloalkyl, R^{1a}-substituted or unsubstituted heterocycloalkyl, R^{1a}-substituted or unsubstituted aryl, or R^{1a}-substituted or unsubstituted heteroaryl.

R^{1a} is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)NH₂, —NO₂, —C(O)H, —C(O)OH, —C(O)NH₂, —OH, —NHSO₂H, —NHC(O)H, —NHC(O)OH, —NHOH, —OCF₃, oxo, —N₃, R^{1b}-substituted or unsubstituted alkyl, R^{1b}-substituted or unsubstituted heteroalkyl, R^{1b}-substituted or unsubstituted cycloalkyl, R^{1b}-substituted or unsubstituted heterocycloalkyl, R^{1b}-substituted or unsubstituted aryl, or R^{1b}-substituted or unsubstituted heteroaryl.

R^{1b} is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

In embodiments, R^{1b} is unsubsituted C₁-C₅ alkyl.

In embodiments, R², R³, R⁴, R⁵, R⁶, R⁷, R⁸ and R⁹ are independently selected from hydrogen, halogen, —CX^{b}₃, —CHX^{b}₂, —CH₂X^{b}, —CN, —SO₂Cl, —SOₙ₂R⁶, —SOₙ₂NR⁷R⁸, —NHNH₂, —ONR⁷R⁸, —NHC═(O)NHNH₂, —NHC═(O)NR⁷R⁸, —N(O)ₘ₂, —NR⁷R⁸, —C(O)R⁹, —C(O)OR⁹, —C(O)NR⁷R⁸, —OR⁶, —NR⁷SO₂R⁶, —NR⁷C(O)R⁹, —NR⁷C(O)OR⁹, —NR⁷OR⁹, —OCX^{b}₃, R¹⁹-substituted or unsubstituted alkyl, R¹⁰-substituted or unsubstituted heteroalkyl, R¹⁰-substituted or unsubstituted cycloalkyl, R¹⁰-substituted or unsubstituted heterocycloalkyl, R¹⁰-substituted or unsubstituted aryl, or R¹⁰-substituted and unsubstituted heteroaryl.

In embodiments, R² and R³ substituents are optionally joined to form R¹⁰-substituted or unsubstituted heterocycloalkyl, or R¹⁰-substituted or unsubstituted heteroaryl and R⁷ and R⁸ substituents are optionally joined to form R¹⁰-substituted or unsubstituted heterocycloalkyl, or R¹⁰-substituted or unsubstituted heteroaryl.

R¹⁰ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)NH₂, —NO₂, —C(O)H, —C(O)OH, —C(O)NH₂, —OH, —NHSO₂H, —NHC(O)H, —NHC(O)OH, —NHOH, —OCF₃, —N₃, oxo, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted and unsubstituted heteroaryl.

R¹¹ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)NH₂, —NO₂, —C(O)H, —C(O)OH, —C(O)NH₂, —OH, —NHSO₂H, —NHC(O)H, —NHC(O)OH, —NHOH, —OCF₃, —N₃, oxo, unsubstituted alkyl, unsubstituted heteroalkyl, unsubstituted cycloalkyl, unsubstituted heterocycloalkyl, unsubstituted aryl, or unsubstituted heteroaryl.

In embodiments, R¹¹ is unsubstituted C₁-C₅ alkyl.

In embodiments, R² and R³ substituents are joined to form R¹⁰-substituted or unsubstituted heterocycloalkyl, or R¹⁰-substituted or unsubstituted heteroaryl

In embodiments, R² and R³ substituents are joined to form R¹⁰-substituted or unsubstituted heterocycloalkyl and R¹⁰ is unsubsituted C₁-C₅ alkyl.

In embodiments, R⁷ and R⁸ substituents are joined to form R¹⁰-substituted or unsubstituted heterocycloalkyl, or R¹⁰-substituted or unsubstituted heteroaryl.

In embodiments, R⁷ and R⁸ substituents are joined to form R¹⁰-substituted or unsubstituted heterocycloalkyl and R¹⁰ is unsubsituted C₁-C₅ alkyl.

In embodiments, R^{1b} is methyl.

In embodiments, R^{1a} is R^{1b}-unsubstituted alkyl.

In embodiments, R^{1a} is R^{1b}-unsubstituted alkyl and z1 is 2

In embodiments, R^{1a} is R^{1b}-unsubstituted alkyl and z1 is 1.

In embodiments, R^{1a} is R^{1b}-unsubstituted alkyl and z2 is 2.

In embodiments R^{1a} is R^{1b}-unsubstituted alkyl and z2 is 1.

In embodiments, a compound of formula (I) and z1 is 0.

In embodiments, a compound of formula (II) and z2 is 0.

### III. Methods of Treatment

The compounds described herein are useful, inter alia, in methods of treating cancer. Such methods include administering to a subject in need thereof an effective amount of a compound having formula (I) or (II), including embodiments and pharmaceutically acceptable salts thereof. In embodiments, the compound is chosen from:

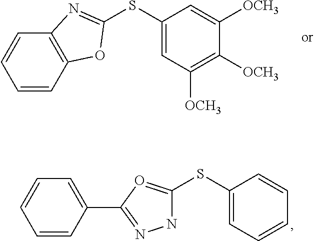

including pharmaceutically acceptable salts thereof.

The cancer may be, for example, lung cancer, breast cancer, ovarian cancer, leukemia, lymphoma, melanoma, pancreatic cancer, sarcoma, bladder cancer, bone cancer, brain cancer, cervical cancer, colon cancer, esophageal cancer, gastric cancer, liver cancer, head and neck cancer, kidney cancer, myeloma, thyroid cancer, or prostate cancer. In embodiments, the cancer is lung cancer, breast cancer, ovarian cancer, leukemia, pancreatic cancer, colon cancer, liver cancer, kidney cancer, prostate cancer, or melanoma. The breast cancer may be hormone sensitive breast cancer or hormone refractory (insensitive) breast cancer. For example the cancer can be ER positive or ER negative breast cancer. In another example, the breast cancer expresses HER-2 and is considered HER-positive. In another example the breast cancer is triple-negative breast cancer.

The compounds described herein are useful for methods of treating cancer triple-negative breast cancer.

Compounds described herein also inhibit cell proliferation in breast cancer cell lines. For example, these breast cancer cell lines may of luminal, basal, HER2 or Claudin-low origin. Examplary cell lines include MCF-7, T47D, SUM185, BT474, ZR-75, MDA-MB-468, SUM190, BT549, MDA-MB-231, Hs578T, SUM1315, SKBR3, MDA-MB-453, BT-483, BT-474, BT-20, AU565, 600MPE, CAMA1, HBL100, HCC1007d, HCC1187d, HCC1143d, HCC1187d, HCC1428d, HCC1500d, HCC1569d, CC1937d, HCC1954d, HCC1954d, HCC2185d, HS578T, LY2, MCF10Ab, MCF12Ab, and DAMB134VI.

### IV. Pharmaceutical Compositions

In another aspect, a pharmaceutical composition including a pharmaceutically acceptable excipient and a compound described herein is provided.

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., inhibiting cell proliferation Determination of a therapeutically effective amount of a compound of the invention is well within the capabilities of those skilled in the art, especially in light of the detailed disclosure herein.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, that may also act as diluents, flavoring agents, binders, preservatives, tablet disintegrating agents, or an encapsulating material.

In powders, the carrier is a finely divided solid in a mixture with the finely divided active component (e.g. a compound provided herein). In tablets, the active component is mixed with the carrier having the necessary binding properties in suitable proportions and compacted in the shape and size desired. The powders and tablets preferably contain from 5% to 70% of the active compound.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragees cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component (e.g. compounds described herein, including embodiments, examples, compounds of Table 1, 2, 3, 4, or 5) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation).

The compounds of the present invention can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compounds of the present invention can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable excipient and one or more compounds of the invention.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. lung cancer, NSCL cancer, colon cancer, colorectal cancer, breast cancer, pancreatic cancer, leukemia), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In one embodiment, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration and dosage can be chosen by the individual physician in view of the patient's condition and the particular method in which the compound is used.

### IV. Examples

Designed herein are new compounds based on the knowledge that diaryl and heteroaryl sulfide containing compounds are useful in many beneficial methods, including therapeutic methods (e.g. treating diseases, such as cancer (e.g. breast cancer)). New and improved methods of making such compounds are therefore of value and are needed.

A mild protocol for the synthesis of diaryl and heteroaryl sulfides is described. In a one-pot procedure, thiols are converted to sulfenyl chlorides and reacted with arylzinc reagents. This method tolerates functional groups including aryl fluorides and chlorides, ketones, as well as N-heterocycles including pyrimidines, imidazoles, tetrazoles, and oxadiazoles (Scheme 1).

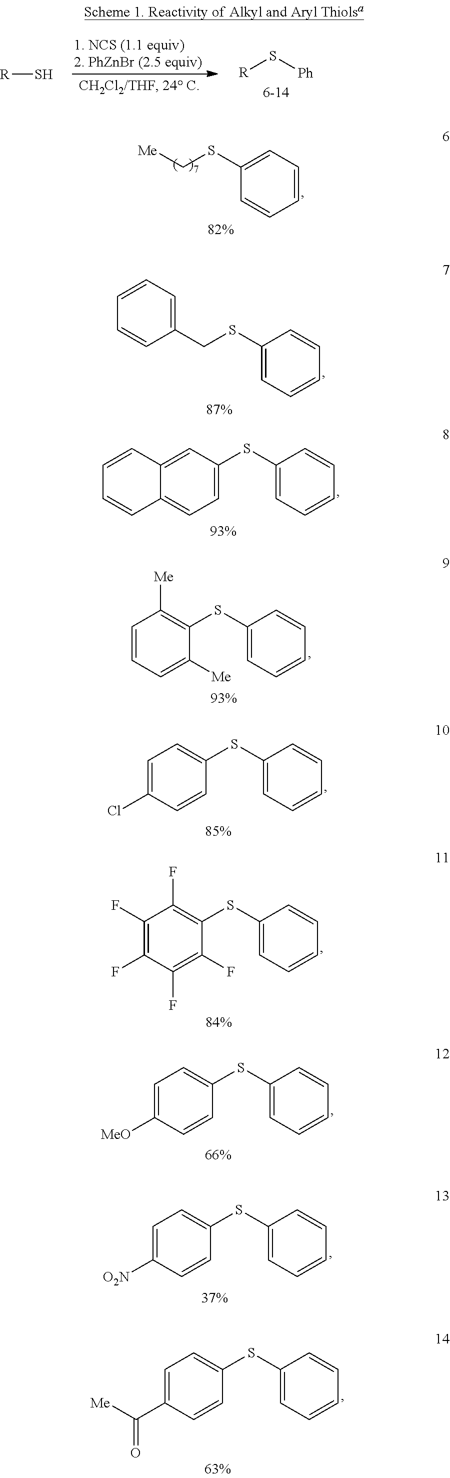

a Isolated yields after silica gel chromatography.

Scheme 1 further shows that a variety of in situ-formed alkyl and aryl sulfenyl chlorides react with phenylzinc bromide to afford the respective thioethers in good yields. Substrates containing ortho-disubstituted aryl rings pose a significant challenge for most metal-catalyzed methods, yet our reaction conditions furnish 9 in 93% yield. Halogenation, and particularly fluorination, is well tolerated. Electron-rich thiols such as 4-methoxy thiophenol are competent in the transformation, although the desired product 12 is afforded in slightly diminished yield due to competitive formation of diaryl disulfide. 4-Nitrothiophenol proved to be a challenging substrate, as it is prone to decomposition under the reaction conditions, thus affording 13 in a modest yield. One benefit of using aryl zinc reagents in contrast to aryl Grignard reagents is the increased functional group compatibility. For example, 1-(4-mercapto-phenyl)-ethanone reacted smoothly to provide desired diaryl sulfide 14 with no observed competitive addition to the ketone.

In an effort to ensure that our method is compatible with the sensitive heterocyclic moieties frequently found in bioactive compounds, several heteroaromatic thiols were examined (Scheme 2). A broad range of heterocycles react with phenylzinc bromide to provide good to excellent yields of the corresponding sulfides,—including benzothiazole, benzoxazole, pyrimidine, tetrazole, oxadiazole, and imidazole functional groups (15-20). Notably, when phenylmagnesium bromide was used instead of phenylzinc bromide, compounds 15, 16, and 20 were obtained in diminished yields (65, 57, and 59% respectively). Furthermore, both electron-rich and electron-poor Grignard reagents react smoothly to afford the desired thioether products in good yields (21 and 22, respectively). meta-Cyanophenylmagnesium reagent is well-tolerated, affording 23 in good yield.

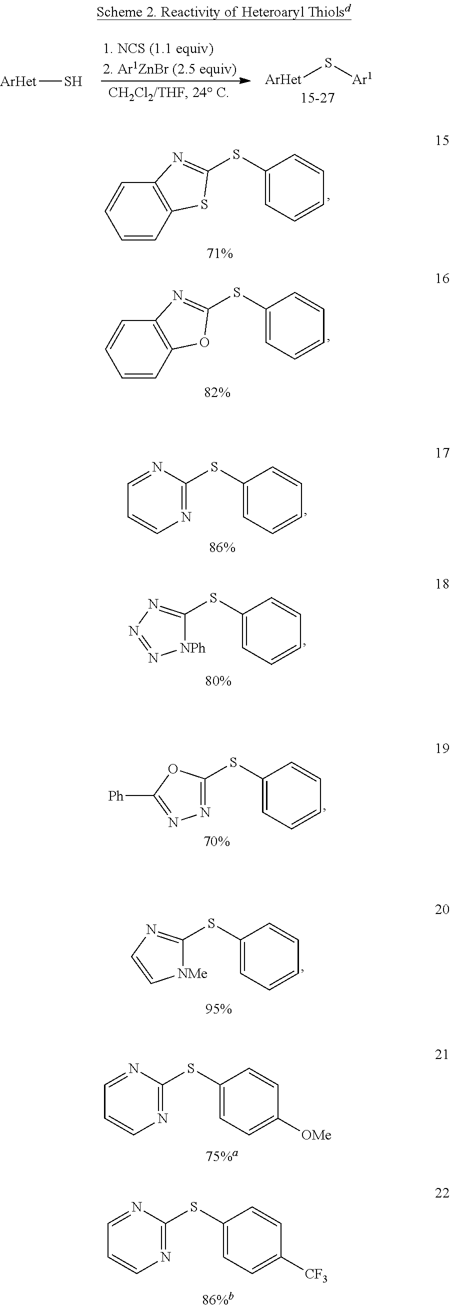

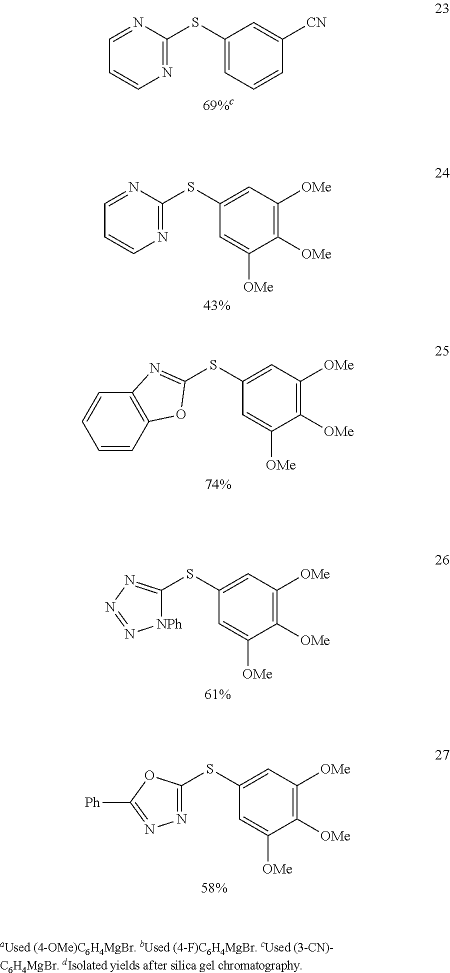

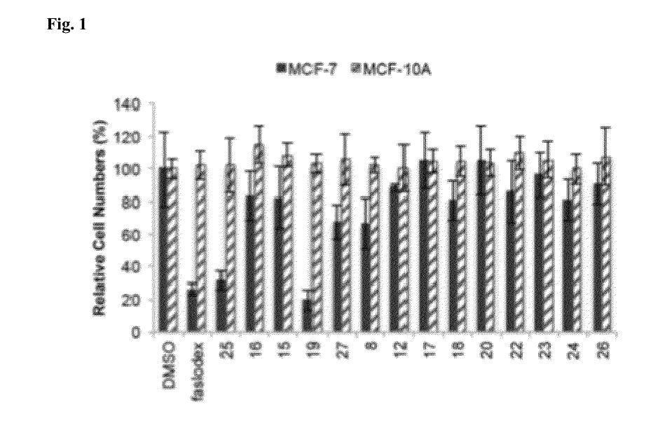

Combretastatin A-4 [1, 2] analogues were prepared as well using this method since it tolerates a diverse range of heterocycles and would further support structure-activity-relationship (SAR) studies of these compounds. Diaryl sulfide analogues of combretastatin containing N-heterocyclic moieties have been reported to be active against MCF-7 breast cancer cell lines (e.g., 2) [3-7]. Reactions of a variety of heteroaryl sulfides with 3,4,5-trimethoxyphenylzinc bromide, biasing our small library of analogues toward inclusion of the 3,4,5-trimethoxyphenyl scaffold, a privileged motif commonly found in anticancer compounds that target microtubules [8, 9] were examined. We were pleased to see that the corresponding arylzinc bromide reacts with a variety of in situ-formed heteroaryl sulfenyl chlorides to afford the respective trimethoxyphenyl-substituted thioethers in modest to good yields (24-27). Having synthesized a variety of combretastatin A-4 analogues, we set out to evaluate these compounds for anti-breast-cancer activity. Select products from Schemes 1 and 2 were tested for anticancer activity against the MCF-7 breast cancer cell line relative to the normal MCF-10A stromal cell line using a proliferation-based procedure (FIG. 1). [10] Results are compared to activity of the estrogen receptor antagonist, faslodex (ICI 182,780) [11, 12] Two compounds demonstrated selective inhibition of cancer cell proliferation. Diaryl sulfide 25, containing benzoxazole and 3,4,5-trimethoxyphenyl moieties, was a potent inhibitor of MCF-7 cell proliferation (EC₅₀=4.5 μM). In comparison, the simple phenyl analogues 16 and 15 were inactive. In contrast, trimethoxyphenyl-containing thioether 27 performed poorly, while its phenyl analogue 19 was a more potent cell proliferation inhibitor (EC₅₀=7.9 μM).

General Procedures.

All reactions were carried out under an atmosphere of N₂ using glassware that was either oven- or flame-dried prior to use. Dichloromethane (CH₂Cl₂) and tetrahydrofuran (THF) were degassed with argon and then passed through two 4×36 in. columns of anhydrous neutral A-2 alumina (8×14 mesh; activated under a flow of argon at 350° C. for 12 h) to remove H₂O. ¹H NMR spectra were recorded on 500 MHz (500 MHz ¹H, 125.7 MHz ¹³C) or 400 MHz (400 MHz IH, 100 MHz ¹³C) spectrometers. Proton chemical shifts are reported in ppm (6) relative to internal tetramethylsilane (TMS, δ 0.00). Data are reported as follows: chemical shift (multiplicity [singlet (s), broad singlet (br s), doublet (d), doublet of doublets (dd), triplet (t), doublet of triplets (dt), quartet (q), multiplet (m), apparent singlet (ap s), and apparent doublet (ap d)], coupling constants [Hz], integration. Carbon chemical shifts are reported in ppm (δ) relative to TMS with the respective solvent resonance as the internal standard (CDCl₃, δ 77.16 ppm). Unless otherwise indicated, NMR data were collected at 25° C. Infrared spectra (thin film or neat) are reported in terms of frequency of absorption (cm⁻¹). Melting points (mp) are uncorrected. Analytical thin-layer chromatography (TLC) was performed using silica gel 60 F₂₅₄ precoated plates (0.25 mm thickness). Visualization was accomplished by irradiation with a UV lamp and/or staining with KMnO₄ solution. Flash chromatography was performed using silica gel 60 Å (170-400 mesh) from Fisher Scientific.

Phenylmagnesium bromide[13] and phenylzinc bromide[14] were prepared according to reported procedures. 4-(Trifluoromethyl)-phenylmagnesium bromide and 4-methoxyphenylmagnesium bromide were prepared from their respective halide precursors in THF. 3-Cyanophenylmagnesium bromide was prepared by magnesium-halogen exchange with isopropylmagnesium bromide in the presence of LiCl. [15] Molarities of organomagnesium and organozinc reagents were determined by titration. [15] N-Chlorosuccinimide (NCS) was recrystallized from benzene and stored in an amber vial for up to two weeks.

### Synthesis 1: General Procedure A for Sulfenylation of Arylzinc Reagents

To a solution of NCS (0.073 g, 0.55 mmol) in DCM (1.0 mL) was added thiol (0.50 mmol), and the solution was stirred for 30 min in the absence of direct light. The solution was taken up using a Teflon needle and added dropwise to a solution of arylzinc reagent in THF (1.25 mmol). Upon completion, as judged by TLC, the reaction mixture was quenched with MeOH and concentrated in vacuo, and the residue was adsorbed onto 3 mL of silica gel and purified by flash column chromatography.

### Synthesis 2: General Procedure B for Sulfenylation of Arylmagnesium Reagents

To a solution of NCS (0.073 g, 0.55 mmol) in DCM (1.0 mL) was added thiol (0.50 mmol), and the solution was stirred for 30 min in the absence of direct light. The solution was taken up using a Teflon needle and added dropwise to a solution of arylmagnesium reagent in THF (1.25 mmol). Upon completion, as judged by TLC, the reaction mixture was quenched with MeOH and concentrated in vacuo, and the residue was adsorbed onto 3 mL of silica gel and purified by flash column chromatography.

### Synthesis 3: Preparation of Octyl(Phenyl)Sulfane (6)

Title compound was prepared according to general procedure A from octane thiol (0.087 mL, 0.50 mmol), NCS (0.073 g, 0.55 mmol) and PhZnBr (1.3 mmol, 1.7 mL). Purification by flash column chromatography (3% EtOAc in hexanes) afforded the title compound as a colorless oil (0.091 g, 82%). Spectral data were consistent with reported values [34]: TLC R_{f}=0.7 (10% EtOAc in hexanes); ¹H NMR (400 MHz, CDCl₃) δ 7.33-7.25 (m, 4H), 7.15 (t, J=7.0 Hz, 1H), 2.91 (t, J=7.3 Hz, 2H), 1.68-1.61 (m, 2H), 1.42 (m, 2H), 1.27 (m, 8H), 0.88 (t, J=6.8 Hz, 3H); ¹³C NMR (125 MHz, CDCl₃) δ 137.2, 128,9 (2C), 125.7, 33.7, 31.9, 29.31, 29.27 (2C), 29.0, 22.8, 14.2.

### Synthesis 4: Preparation of Benzyl(Phenyl)Sulfane (7)

Title compound was prepared according to general procedure A from benzyl mercaptan (0.059 mL, 0.50 mmol), NCS (0.073 g, 0.55 mmol) and PhZnBr (1.3 mmol, 1.7 mL). Purification by flash column chromatography (3% EtOAc in hexanes) afforded the title compound as a colorless oil (0.087 g, 87%). Spectral data were consistent with reported values [17]: TLC R_{f}=0.5(10% EtOAc in hexanes); ¹H NMR (400 MHz, CDCl₃) δ 7.31-7.18 (m, 9H), 7.16 (t, J=7.2 Hz, 1H), 4.10 (s, 2H); ¹³C NMR (125 MHz, CDCl₃) δ 137.6, 136.5, 129.9, 128.95, 128.94, 128.6, 127.3, 126.4, 39.1.

### Synthesis 5: Preparation of 2-Napthyl(phenyl)sulfane (8)

Title compound was prepared according to general procedure A from thio-2-naphthol (0.080 g, 0.50 mmol), NCS (0.073 g, 0.55 mmol) and PhZnBr (1.3 mmol, 1.7 mL). Purification by flash column chromatography (3% EtOAc in hexanes) afforded the title compound as a colorless oil (0.110 g, 93%). Spectral data were consistent with reported values [16]: TLC R_{f}=0.6 (10% EtOAc in hexanes); ¹H NMR (400 MHz, CDCl₃) δ 7.81 (s, 1H), 7.75-7.66 (m, 3H), 7.44-7.34 (m, 5H), 7.28-7.18 (m, 3H); ¹³C NMR (125 MHz, CDCl₃) δ 136.0, 133.9, 133.1, 132.4, 131.0, 130.0, 129.3, 129.0, 128.8, 127.8, 127.5, 127.1, 126.7, 126.3.

### Synthesis 6: Preparation of 2,6-Dimethylphenyl(phenyl)sulfane (9)

Title compound was prepared according to general procedure A from 2,6-dimethylthiophenol (0.069 mL, 0.50 mmol), NCS (0.073 g, 0.55 mmol) and PhZnBr (1.3 mmol, 1.7 mL). Purification by flash column chromatography (3% EtOAc in hexanes) afforded the title compound as a colorless oil (0.100 g, 93%). Spectral data were consistent with reported values: [18] TLC R_{f}=0.5 (10% EtOAc in hexanes); ¹H NMR (400 MHz, CDCl₃) δ 7.22-7.14 (m, 5H), 7.04 (t, J=7.2 Hz, 1H), 6.92 (d, J=7.6 Hz, 2H), 2.42 (s, 6H); ¹³C NMR (125 MHz, CDCl₃) δ 144.0, 138.1, 130.6, 129.4, 129.0, 128.6, 125.8, 124.7, 22.0.

### Synthesis 7: Preparation of 4-Chlorophenyl(phenyl)sulfane (10)

Title compound was prepared according to general procedure A from 4-chlorobenzenethiol (0.072 g, 0.50 mmol), NCS (0.073 g, 0.55 mmol) and PhZnBr (1.3 mmol, 1.7 mL). Purification by flash column chromatography (3% EtOAc in hexanes) afforded the title compound as a colorless oil (0.094 g, 85%). Spectral data were consistent with reported values: [16] TLC R_{f}=0.7 (10% EtOAc in hexanes); ¹H NMR (400 MHz, CDCl₃) δ 7.35-7.29 (m, 4H), 7.28-7.22 (m, 5H); ¹³C NMR (100 MHz, CDCl₃) δ 135.2, 134.8, 133.1, 132.1, 131.4, 129.5, 129.4, 127.6.

### Synthesis 8: Preparation of (Perfluorophenyl)(phenyl)sulfane (11)

Title compound was prepared according to general procedure A from pentafluorothiophenol (0.067 mL, 0.50 mmol), NCS (0.073 g, 0.55 mmol) and PhZnBr (1.3 mmol, 1.7 mL). Purification by flash column chromatography (3% EtOAc in hexanes) afforded the title compound as a colorless crystalline solid (0.114 g, 84%). Spectral data were consistent with reported values: [19] TLC R_{f}=0.6 (5% EtOAc in hexanes); mp 45-48° C.; ¹H NMR (400 MHz, CDCl₃) δ 7.35 (m, 2H), 7.32-7.24 (m, 3H); ¹³C NMR (125 MHz, CDCl₃) δ 148.7 (m), 146.7 (m), 143.2 (m), 141.2 (m), 139.0 (m), 136.9 (m), 133.1, 130.7, 129.6, 128.1, 109.1 (m); ¹⁹F NMR (376 MHz, CDCl₃) 6-131.9 (dd, J=24.7 Hz, 7.0 Hz, 2F), —151.6 (t, J=20.9 Hz, 1F), —160.6 (td, J=22.2 Hz, 6.7 Hz, 2F); IR (neat) 1482, 1093, 971 cm⁻¹; HRMS (TOF MS CI+) m/z calcd for C₁₂H₅F₅S (M)⁺276.0032. found 276.0025.

### Synthesis 9: Preparation of (4-Methoxyphenyl)(phenyl)sulfane (12)

Title compound was prepared according to general procedure A from 4-methoxybenzenethiol (0.062 mL, 0.50 mmol), NCS (0.073 g, 0.55 mmol) and PhZnBr (1.3 mmol, 1.7 mL). Purification by flash column chromatography (10% EtOAc in hexanes) afforded the title compound as a colorless oil (0.071 g, 66%). Spectral data were consistent with reported values: [16] TLC R_{f}=0.5 (10% EtOAc in hexanes); ¹H NMR (400 MHz, CDCl₃) δ 7.40 (d, J=8.5 Hz, 2H), 7.22-7.11 (m, 5H), 6.87 (d, J=8.6 Hz, 2H), 3.77 (s, 3H); ¹³C NMR (125 MHz, CDCl₃) δ 159.8, 138.7, 135.4, 129.0, 128.2, 125.8, 124.3, 115.0, 55.3.

### Synthesis 10: Preparation of (4-Nitrophenyl)(phenyl)sulfane (13)

Title compound was prepared according to general procedure A from 4-nitrobenzenethiol (0.082 g, 0.50 mmol), NCS (0.073 g, 0.55 mmol) and PhZnBr (1.3 mmol, 1.7 mL). Purification by flash column chromatography (10% EtOAc in hexanes) afforded the title compound as a colorless oil (0.042 g, 37%). Spectral data were consistent with reported values: [16] TLC R_{f}=0.4 (5% EtOAc in hexanes); 1H NMR (400 MHz, CDCl₃) δ 8.06 (dt, J=9.6 Hz, J=2.2 Hz, 2H), 7.53 (m, 2H), 7.46 (m, 3H), 7.18 (dt, J=9.6 Hz, J=2.2 Hz, 2H); ¹³C NMR (125 MHz, CDCl₃) δ 148.6, 145.4, 134.8, 130.5, 130.1, 129.7, 126.7, 124.1.

### Synthesis 11: Preparation of 4-Phenylsulfanylacetophenone (14)

Title compound was prepared according to general procedure A from 1-(4-sulfanylphenyl)-ethan-1-one (60 μL, 0.5 mmol), NCS (0.073 g, 0.55 mmol) and PhZnBr (1.3 mmol, 2.6 mL). Purification by flash column chromatography (5% EtOAc in hexanes) afforded the title compound as a pale yellow solid (0.072 g, 63%). Spectral data were consistent with reported values: [20] TLC R_{f}=0.3 (5% EtOAc in hexanes); ¹H NMR (400 MHz, CDCl₃) δ 7.81 (d, J=8.5 Hz, 2H), 7.50-7.47 (m, 2H), 7.40-7.38 (m, 3H), 7.20 (d, J=8.5 Hz, 2H), 2.54 (s, 3H); ¹³C NMR (125 MHz, CDCl₃) δ 197.3, 145.0, 134.5, 134.0, 132.1, 129.8, 129.0, 128.9, 127.5, 26.5; IR (neat) 2922, 1677, 1589, 690 cm⁻¹.

### Synthesis 12: Preparation of 2-Phenylthiobenzothiazole (15)

Title compound was prepared according to general procedure A from 2-mercaptobenzothiazole (0.084 g, 0.50 mmol), NCS (0.073 g, 0.55 mmol) and PhZnBr (1.3 mmol, 1.7 mL). Purification by flash column chromatography (15% EtOAc in hexanes) afforded the title compound as a colorless oil (0.087 g, 71%). Compound 15 was also prepared from PhMgBr according to general procedure B to afford 65% yield (determined by ¹H NMR in comparison to the internal standard phenyltrimethylsilane). Spectral data were consistent with reported values: [21] TLC R_{f}⁼0.5 (30% EtOAc in hexanes); ¹H NMR (400 MHz, CDCl₃) δ 7.87 (d, J=8.4 Hz, 1H), 7.72 (m, 2H), 6.63 (d, J=8.0 Hz, 1H), 7.52-7.43 (m, 3H), 7.38 (m, 1H), 7.25 (m, 1H); ¹³C NMR (125 MHz, CDCl₃) δ 169.8, 154.0, 135.6, 135.4, 130.6, 130.01, 129.98, 126.2, 124.4, 122.0, 120.9.

### Synthesis 13: Preparation of 2-Phenylthiobenzoxazole (16)

Title compound was prepared according to general procedure A from 2-mercaptobenzoxazole (0.076 g, 0.50 mmol), NCS (0.073 g, 0.55 mmol) and PhZnBr (1.3 mmol, 1.7 mL. Purification by flash column chromatography (15% EtOAc in hexanes) afforded the title compound as a colorless oil (0.093 g, 82%). Compound 16 was also prepared from PhMgBr according to general procedure B to afford 57% yield (determined by ¹H NMR in comparison to the internal standard phenyltrimethylsilane). Spectral data were consistent with reported values:³⁹ TLC R_{f}=0.5 (30% EtOAc in hexanes); ¹H NMR (500 MHz, CDCl₃) δ 7.70 (m, 2H), 7.59 (m, ¹H), 7.47-7.42 (m, 3H), 7.39 (m, 1H), 7.27-7.21 (m, 2H); ¹³C NMR (100 MHz, CDCl₃) δ 163.4, 152.0, 142.1, 134.5, 130.0, 129.8, 127.3, 124.5, 124.4, 119.2, 110.2.

### Synthesis 14: Preparation of 2-(Phenylthio)pyrimidine (17)

Title compound was prepared according to general procedure A from 2-mercaptopyrimidine (0.056 g, 0.50 mmol), NCS (0.073 g, 0.55 mmol) and PhZnBr (1.3 mmol, 1.7 mL). Purification by flash column chromatography (20% EtOAc in hexanes) afforded the title compound as a colorless oil (0.081 g, 86%). Spectral data were consistent with reported values:⁴⁰ TLC R_{f}=0.3 (30% EtOAc in hexanes); ¹H NMR (500 MHz, CDCl₃) δ 8.47 (d, J=5.0 Hz, 2H), 7.63 (m, 2H), 7.44 (m, 3H), 6.95 (t, J=5.0 Hz, 1H); ¹³C NMR (125 MHz, CDCl₃) δ 172.9, 157.7, 135.4, 129.45, 129.43, 129.3, 117.1.

### Synthesis 15: Preparation of 1-Phenyl-5-(phenylthio)-1H-tetrazole (18)

Title compound was prepared according to general procedure A from 1-phenyl-1H-tetrazole-5-thiol (0.089 g, 0.50 mmol), NCS (0.073 g, 0.55 mmol) and PhZnBr (1.3 mmol, 1.7 mL). Purification by flash column chromatography (30% EtOAc in hexanes) afforded the title compound as a colorless crystalline solid (0.102 g, 80%): TLC R_{f}=0.4 (30% EtOAc in hexanes); mp 129-133° C.; ¹H NMR (400 MHz, CDCl₃) δ 7.57-7.53 (m, 7H), 7.42-7.36 (m, 3H); ¹³C NMR (125 MHz, CDCl₃) δ 153.7, 134.0, 133.7, 130.5, 130.1, 129.86, 129.85, 126.9, 124.5; IR (neat) 3067, 2922, 1498, 1412, 1389, 1240 cm⁻¹; HRMS (TOF MS ES+) m/z calcd for C₁₃H₁₀N₄S (M+Na)⁺277.0524. found 277.0524.

### Synthesis 16: Preparation of 2-Phenyl-5-(phenylthio)-1,3,4-oxadiazole (19)

Title compound was prepared according to general procedure A from 5-phenyl-1,3,4-oxadiazaole-2-thiol (0.089 g, 0.50 mmol), NCS (0.073 g, 0.55 mmol) and PhZnBr (1.3 mmol, 1.7 mL). Purification by flash column chromatography (20-50% EtOAc in hexanes) afforded the title compound as a white solid (0.089 g, 70%). Spectral data were consistent with reported values:⁴¹ TLC R_{f}=0.5 (20% EtOAc in hexanes); ¹H NMR (500 MHz, CDCl₃) δ 7.94 (d, J=7.0 Hz, 2H), 7.67 (m, 2H), 7.51-7.39 (m, 6H); ¹³C NMR (125 MHz, CDCl₃) δ 166.4, 162.9, 133.7, 131.9, 129.9, 129.8, 129.1, 127.1, 126.8, 123.5.

### Synthesis 17: Preparation of 1-Methyl-2-(phenylthio)-1H-imidazole (20)

Title compound was prepared according to general procedure A from 2-mercapto-1-methylimidazole (0.057 g, 0.50 mmol), NCS (0.073 g, 0.55 mmol) and PhZnBr (1.3 mmol, 1.7 mL). Purification by flash column chromatography (10% EtOAc in hexanes) afforded the title compound as a colorless oil (0.081 g, 95%). Compound 20 was also prepared from PhMgBr according to general procedure B to afford 59% yield (determined by ¹H NMR in comparison to the internal standard phenyltrimethylsilane). Spectral data were consistent with reported values:³⁹ TLC R_{f}=0.2 (30% EtOAc in hexanes); ¹H NMR (500 MHz, CDCl₃) δ 7.25 (m, 2H), 7.18-7.13 (m, 4H), 7.06 (d, J=1.0 Hz, 1H), 3.62 (s, 3H); ¹³C NMR (125 MHz, CDCl₃) δ 138.1, 135.0, 130.2, 129.3, 128.0, 126.6, 123.9, 33.9.

### Synthesis 18: Preparation of 2-(4-Methoxyphenylthio)pyrimidine (21)

Title compound was prepared according to general procedure B from 2-mercaptopyr-imidine (0.056 g, 0.50 mmol), NCS (0.073 g, 0.55 mmol) and (4-OMe)PhMgBr (1.3 mmol, 1.8 mL). Purification by flash column chromatography (20-30% EtOAc in hexanes) afforded the title compound as a white solid (0.082 g, 75%). Spectral data were consistent with reported values:⁴⁰ TLC R_{f}=0.4 (30% EtOAc in hexanes); ¹H NMR (400 MHz, CDCl₃) δ 8.47 (d, J=4.4 Hz, 2H), 7.54 (d, J=8.0 Hz, 2H), 6.95 (m, 3H), 3.83 (s, 3H); ¹³C NMR (100 MHz, CDCl₃) δ 173.5, 160.6, 157.6, 137.1, 120.0, 116.8, 114.9, 55.4.

### Synthesis 19: Preparation of 2-(4-(Trifluoromethyl)phenylthio)pyrimidine (22)

Title compound was prepared according to general procedure B from 2-mercaptopyrimidine (0.056 g, 0.50 mmol), NCS (0.073 g, 0.55 mmol) and (4-CF3)PhMgBr (1.3 mmol, 2.1 mL). Purification by flash column chromatography (10-30% EtOAc in hexanes) afforded the title compound as a colorless oil (0.110 g, 86%): TLC R_{f}=0.6 (30% EtOAc in hexanes); ¹H NMR (400 MHz, CDCl₃) δ 8.51 (d, J=4.8 Hz, 2H), 7.76 (d, J=8.0 Hz, 2H), 7.67 (d, J=8.0 Hz, 2H), 7.02 (t, J=4.8 Hz, 1H); ¹³C NMR (100 MHz, CDCl₃) δ 171.7, 157.8, 135.1, 134.6 (ap d, J=1.4 Hz, 1C), 131.1 (q, J=32.8 Hz, 1C), 126.1 (q, J=3.7 Hz, 1C), 124.0 (q, J=272.4 Hz, 1C), 117.6; ¹⁹F NMR (376 MHz, CDCl₃) δ −63.0; IR (thin film) 3039, 2927, 1566, 1389, 1329, 1170, 1122 cm⁻¹; HRMS (TOF MS CI+) m/z calcd for C₁₁H⁷F₃N₂S (M+H)⁺257.0360. found 257.0353.

### Synthesis 20: Preparation of 3-(Pyrimidin-2-ylthio)benzonitrile (23)

Title compound was prepared according to general procedure B from 2-mercaptopyrimidine (0.056 g, 0.50 mmol), NCS (0.073 g, 0.55 mmol) and (3-CN)PhMgBr (1.3 mmol, 2.3 mL. Purification by flash column chromatography (10-30% EtOAc in hexanes) afforded the title compound as a white solid (0.074 g, 69%): TLC R_{f}=0.4 (30% EtOAc in hexanes); mp 71-73° C.; 1H NMR (400 MHz, CDCl₃) δ 8.51 (d, J=4.4 Hz, 2H), 7.95 (s, 1H), 7.86 (d, J=8.0 Hz, 1H), 7.71 (d, J=7.6 Hz, 1H), 7.55 (t, J=7.6 Hz, ¹H), 7.06 (t, J=4.2 Hz, 1H); ¹³C NMR (100 MHz, CDCl₃) δ 171.1, 157.7, 139.2, 138.3, 132.5, 131.7, 129.8, 118.1, 117.7, 113.4; IR (thin film) 3066, 2927, 2231, 1560, 1379, 1182 cm⁻¹; HRMS (TOF MS CI+) m/z calcd for C₁₁H₇N₃S (M+H)⁺214.0439. found 214.0433.

### Synthesis 21: Preparation of 2-(3,4,5-Trimethoxyphenylthio)pyrimidine (24)

Title compound was prepared according to general procedure A from 2-mercaptopyrimidine (0.056 g, 0.50 mmol), NCS (0.073 g, 0.55 mmol) and 3,4,5-trimethoxyphenylzinc bromide (1.3 mmol, 2.8 mL). Purification by flash column chromatography (20-50% EtOAc in hexanes, 1% Et3N) afforded the title compound as a white solid (0.059 g, 43%): TLC R_{f}=0.1 (30% EtOAc in hexanes); mp 103-104° C.; ¹H NMR (400 MHz, CDCl₃) δ 8.52 (d, J=4.8 Hz, 2H), 6.99 (t, J=4.8 Hz, 1H), 6.88 (s, 2H), 3.90 (s, 3H), 3.87 (s, 6H); ¹³C NMR (125 MHz, CDCl₃) δ 173.1, 157.8, 153.6, 139.2, 123.8, 117.1, 112.4, 61.0, 56.3; IR (neat) 2945, 2851, 1547, 1375, 1117 cm-1; HRMS (TOF MS ES+) m/z calcd for C₁₃H₁₄N₂O₃S (M+Na)⁺301.0623. found 301.0616.

### Synthesis 22: Preparation of 2-(3,4,5-Trimethoxyphenylthio)benzoxazole (25)

Title com-pound was prepared according to general procedure A from 2-mercaptobenzoxazole (0.076 g, 0.50 mmol), NCS (0.073 g, 0.55 mmol) and 3,4,5-trimethoxyphenylzinc bromide (1.3 mmol, 2.8 mL). Purification by flash column chromatography (5-15% EtOAc in hexanes, 1% Et₃N) afforded the title compound as a white solid (0.117 g, 74%): TLC Rf=0.4 (30% EtOAc in hexanes); mp 129-130 ‘C; ¹H NMR (400 MHz, CDCl₃) δ 7.62 (dd, J=7.6 Hz, J=5.6 Hz, 1H), 7.44 (dd, J=8.8 Hz, J=6.0 Hz, 1H), 7.27 (m, 2H), 6.94 (s, 2H), 3.90 (s, 3H), 3.88 (s, 6H); ¹³C NMR (125 MHz, CDCl₃) δ 163.6, 153.8, 152.0, 142.1, 139.8, 124.54, 124.45, 121.2, 119.2, 112.0, 110.2, 61.0, 56.4; IR (neat) 2931, 2837, 1489, 1451, 1406, 1232, 1129, 1121 cm-1; HRMS (TOF MS ES+) m/z calcd for C₁₆H₁₅NO₄S (M+Na)⁺340.0620. found 340.0620.

### Synthesis 23: Preparation of 1-Phenyl-5-(3,4,5-trimethoxyphenylthio)-1H-tetrazole (26)

Title compound was prepared according to general procedure A from 1-phenyl-1H-tetrazole-5-thiol (0.089 g, 0.50 mmol), NCS (0.073 g, 0.55 mmol) and 3,4,5-trimethoxyphenylzinc bromide (1.3 mmol, 2.8 mL). Purification by flash column chromatography (30-50% EtOAc in hexanes, 1% Et₃N) afforded the title compound as a white solid (0.105 g, 61%): TLC R_{f}=0.3 (30% EtOAc in hexanes); mp 110-111° C.; ¹H NMR (400 MHz, CDCl₃) 67.57 (m, 5H), 6.78 (s, 2H), 3.86 (s, 3H), 3.82 (s, 6H); ¹³C NMR (125 MHz, CDCl₃) δ 153.9, 153.8, 139.9, 133.8, 130.5, 129.9, 124.7, 120.7, 111.7, 61.0, 56.4; IR (neat) 3042, 2951, 2860, 1585, 1408, 1231, 1125 cm⁻¹; HRMS (TOF MS ES+) m/z calcd for C₁₆H₁₆N₄O₃S (M+Na)⁺367.0841. found 367.0836.

### Synthesis 24: Preparation of 2-Phenyl-5-(3,4,5-trimethoxyphenylthio)-1,3,4-oxadiazole (27)

Title compound was prepared according to general procedure A from 5-phenyl-1,3,4-oxadiazaole-2-thiol (0.089 g, 0.50 mmol), NCS (0.073 g, 0.55 mmol) and 3,4,5-trimethoxyphenylzinc bromide (1.3 mmol, 2.8 mL). Purification by flash column chromatography (5-25% EtOAc in hexanes, 1% Et₃N) afforded the title compound as a white solid (0.100 g, 58%): TLC R_{f}=0.3 (30% EtOAc in hexanes); mp 142° C.; ¹H NMR (400 MHz, CDCl₃) δ 7.98 (d, J=6.8 Hz, 2H), 7.51 (m, 3H), 6.93 (s, 2H), 3.88 (s, 9H); ¹³C NMR (100 MHz, CDCl₃) δ 166.5, 163.3, 153.9, 139.8, 132.0, 129.2, 126.9, 123.6, 121.0, 111.5, 61.1, 56.5; IR (neat) 3009, 2943, 2850, 1582, 1463, 1128 cm-1; HRMS (TOF MS ES+) m/z calcd for C₁₇H₁₆N₂O₄S (M+Na)⁺367.0728. found 367.0722.

### Synthesis 25: Preparation of General Procedures for Biological Experiments

Biological experiments were performed according to a modified procedure by Sigman et al. [10]

Materials.

The following reagents were obtained from commercial sources as indicated: Dulbecco's Modified Eagle's Medium (DMEM)/high glucose containing 4.5 g/L glucose and 4.0 mM L-glutamine (HyClone); fetal bovine serum (FBS), heat-inactivated (Omega Scientific); L-glutamine, 200 mM (Gibco); penicillin/streptomycin solution SOX (Mediatech); DMEM/Ham's Nutrient Mixture F12 containing 2.5 mM L-glutamine, 3151 mg/L dextrose, and 55 mg/L sodium pyruvate (Sigma-Aldrich); horse serum (Sigma-Aldrich); 50 μM hydrocortisone solution (Sigma-Aldrich); human insulin solution (Sigma-Aldrich); cholera toxin (Sigma-Aldrich); human Epidermal Growth Factor (EGF), recombinant (Sigma-Aldrich); 0.25% Trypsin-EDTA (Gibco); nuclease-free sterile water (Fisher Scientific); molecular biology grade DMSO (Sigma-Aldrich); ICI 182,780 (faslodex) (Tocris Bioscience).

Cell Lines and Culture Conditions.

MCF-7 cells were maintained in DMEM/high glucose supplemented with 10% FBS, L-glutamine, and penicillin/streptomycin. Experiments with MCF-7 cells were performed in DMEM/high glucose supplemented with 2% FBS, L-glutamine, and penicillin/streptomycin. MCF-10A cells were maintained in standard medium according to a modified recipe by Brugge et al. [22] DMEM/F12 supplemented with 5% horse serum, 10 μg/mL of human insulin, 0.5 μg/mL of hydrocortisone, 10 ng/mL of EGF, 100 ng/mL of cholera toxin, and penicillin/streptomycin. Experiments with MCF-10A cells were performed in the same medium.

Evaluation of Compounds Against MCF-7 Cells.

MCF-7 cells were centrifuged in 1×PBS for 20 min, and then the pellet was resuspended in DMEM supplemented with 10% FBS and filtered through a 40 μm nylon cell strainer (Fisher Scientific) to prevent clumping. The cells were seeded at 1500 cells per well in 96-well flat bottom plates suitable for fluorimetry, using 175 μL per well of DMEM supplemented with 10% FBS, and grown for 24 h in 5% CO₂ at 37° C. The compounds (including the faslodex positive control) were dissolved in molecular biology grade DMSO to achieve a 3.5 mM stock solution and then sterile filtered through a 0.45 μm PVDF syringe filter unit (Fisher Scientific). The 3.5 mM stock solutions were subsequently diluted to a final concentration of 10 μM in DMEM supplemented with 2% FBS. Additionally, the corresponding DMSO vehicle control was diluted using the same medium.

After 24 h of growth, the cells were treated by replacing the normal media with fresh media containing the individual compounds or vehicle control (day 0). The outer rows of wells were not used to eliminate the possibility of effects due to evaporation of media. The cells were incubated with compound for 48 h and then treated again by aspirating the media and adding fresh media containing the compounds and controls (day 2). This procedure was repeated after an additional 48 h (day 4). After incubating a final 24 h, the 96-well plates were rinsed with 1×PBS, blotted dry, and then frozen at −78° C. overnight (day 5). On day 6, cell proliferation was measured using the fluorescence-based CyQUANT Cell Proliferation Assay Kit (Invitrogen).

Fluorimetry analysis was performed according to a modified procedure by McGowan et al. [23] Cells were stained with 200 μL/well of 1× CyQUANT GR dye in cell lysis buffer for 10 min in the dark at room temperature and quantified by fluorimetry at 535 nm with 485 nm excitation. The fluorescence values were normalized to the DMSO vehicle control. The normalized values were plotted as an average±standard deviation of 6 wells per compound.

Evaluation of Compounds Against MCF-10A Cells.

MCF-10A cells were centrifuged in 1×PBS for 20 min, and then the pellet was resuspended in DMEM/F12 and filtered through a 40 μm nylon cell strainer (Fisher Scientific) to prevent clumping. The cells were seeded at 9000 cells per well in 96-well flat bottom plates suitable for fluorimetry, using 175 μL, per well of DMEM/F12, and grown for 24 h in 5% CO₂ at 37° C. The 3.5 mM stock solutions of compound in DMSO were subsequently diluted to a final concentration of 10 μM in DMEM/F12. Additionally, the corresponding DMSO vehicle control was diluted using the same medium.

Addition of compounds was performed as specified above for days 0-6. Fluorimetry analysis was performed as specified above for MCF-7 cells, with the exception of staining MCF-10A cells with 200 μL/well of 5× CyQUANT GR dye in cell lysis buffer for 10 min in the dark at room temperature before quantification by fluorimetry. The fluorescence values were normalized to the DMSO vehicle control. The normalized values were plotted as an average±standard deviation of 6 wells per compound.

Dose-Response of Compounds 19 and 25

MCF-7 cells were centrifuged in 1×PBS for 20 min, and then the pellet was resuspended in DMEM supplemented with 10% FBS and filtered through a 40 μm nylon cell strainer (Fisher Scientific) to prevent clumping. The cells were seeded at 1500 cells per well in 96-well flat bottom plates suitable for fluorimetry, using 175 μL, per well of DMEM supplemented with 10% FBS, and grown for 24 h in 5% CO₂ at 37° C. The compounds 19 and 25 were dissolved in molecular biology grade DMSO to achieve a 42 mM stock and then sterile filtered through a 0.45 μm PVDF syringe filter unit (Fisher Scientific). The 42 mM stock solutions in DMSO were subsequently diluted to 120 μM in DMEM supplemented with 2% FBS and then serially diluted to achieve 10 different concentrations. Additionally, the corresponding DMSO vehicle controls for each concentration were serially diluted using the same medium.

Addition of compounds was performed as specified above for days 0-6. Fluorimetry analysis was performed as specified above for the evaluation of compounds against MCF-7 cells. The fluorescence values were normalized to the DMSO vehicle controls corresponding to each concentration. The normalized values were plotted as an average±standard deviation of 4 wells per concentration, and these data were analyzed using the dose-response nonlinear regression fitting function (log[inhibitor] vs response with variable slope (four parameters)).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

(1) Lin, C. M.; Ho, H. H.; Pettit, G. R; Hamel, E. Biochemistry 1989, 28, 6984.
(2) Tron, G. C.; Pirali, T.; Sorba, G.; Pagliai, F.; Busacca, S.; Genazzani, A. A. J. Med. Chem. 2006, 49, 3033.
(3) De Martino, G.; La Regina, G.; Coluccia, A.; Edler, M. C.; Barbera, M. C.; Brancale, A.; Wilcox, E.; Hamel, E.; Artico, M.; Silvestri, R J. Med. Chem. 2004, 47, 6120.
(4) Barbosa, E. G.; Bega, L. A. S.; Beatriz, A.; Sarkar, T.; Hamel, E.; do Amaral, M. S.; de Lima, D. P. Eur. J. Med. Chem. 2009, 44, 2685.
(5) La Regina, G.; Bai, R; Rensen, W. M.; Di Cesare, E.; Coluccia, A.; Piscitelli, F.; Famiglini, V.; Reggio, A.; Nalli, M.; Pelliccia, S.; Da Pozzo, E.; Costa, B.; Granata, I.; Porta, A.; Maresca, B.; Soriani, A.; Iannitto, M. L.; Santoni, A.; Li, J.; Cona, M. M.; Chen, F.; Ni, Y.; Brancale, A.; Dondio, G.; Vultaggio, S.; Varasi, M.; Mercurio, C.; Martini, C.; Hamel, E.; Lavia, P.; Novellino, E.; Silvestri, R. J. Med. Chem. 2013, 56, 123.
(6) Lu, Y.; Li, C.-M.; Wang, Z.; Chen, J.; Mohler, M. L.; Li, W.; Dalton, J. T.; Miller, D. D. J. Med. Chem. 2011, 54, 4678.
(7) Lee, H.-Y.; Chang, J.-Y.; Nien, C.-Y.; Kuo, C.-C.; Shih, K-H.; Wu, C.-H.; Chang, C.-Y.; Lai, W.-Y.; Liou, J.-P. J. Med. Chem. 2011, 54, 8517.
(8) Jordan, A.; Hadfield, J. A.; Lawrence, N. J.; McGown, A. T. Med. Res. Rev. 1998, 18, 259.
(9) Messaoudi, S.; Hamze, A.; Provot, O.; Treguier, B.; De Losada, J. R; Bignon, J.; Liu, J.-M.; Wdzieczak-Bakala, J.; Thoret, S.; Dubois, J.; Brion, J.-D.; Alami, M. ChemMedChem 2011, 6, 488.
(10) Pathak, T. P.; Gligorich, K. M.; Welm, B. E.; Sigman, M. S. J. Am. Chem. Soc. 2010, 132, 7870.
(11) Howell, A. Endocr.-Relat. Cancer 2006, 13, 689.
(12) Wakeling, A. E.; Dukes, M.; Bowler, J. Cancer Res. 1991, 51, 3867.
(13) Bollmann, A.; Blann, K; Dixon, J. T.; Hess, F. M.; Killian, E.; Maumela, H.; McGuinness, D. S.; Morgan, D. H.; Neveling, A.; Otto, S.; Overett, M.; Slawin, A. M. Z.; Wasserscheid, P.; Kuhlmann, S J Am. Chem. Soc. 2004, 126, 14712.
(14) Berman, A. M.; Johnson, J. S. Synlett 2005, 1799.
(15) Krasovskiy, A.; Knochel, P. Synthesis 2006, 890.
(16) Swapna, K.; Murthy, S. N.; Jyothi, M.; Nageswar, Y. V. D. Org. Biomol. Chem. 2011, 9, 5989.
(17) Prasad, D. J. C.; Sekar, G. Synthesis 2010, 79.
(18) Murata, M.; Buchwald, S. L. Tetrahedron 2004, 60, 7397.
(19) Yu, C.; Zhang, C.; Shi, X. Eur. J. Org. Chem. 2012, 1953.
(20) Park, N.; Park, K; Jang, M.; Lee, S. J. Org. Chem. 2011, 76, 4371.
(21) Zhou, A.-X.; Liu, X.-Y.; Yang, K; Zhao, S.-C.; Liang, Y.-M. Org. Biomol. Chem. 2011, 9, 5456.
(22) Debnath, J.; Muthuswamy, S. K; Brugge, J. S. Methods 2003, 30, 256.
(23) McGowan, E. M.; Alling, N.; Jackson, E. A.; Yagoub, D.; Haass, N. K; Allen, J. D.; Martinello-Wilks, R. PLoS One 2011, 6, e20623.

What is claimed is:

1. A compound having formula: 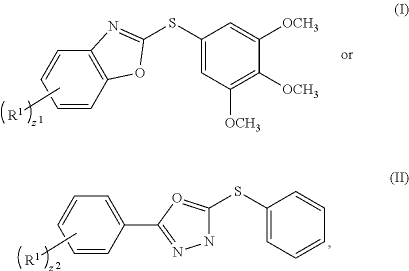
wherein
R¹ is independently a halogen, —NR²R³, —CX^{a}₃, —CHX^{a}₂, —CH₂X^{a}, —CN, —SO₂Cl, —SOₙ₁R⁴, —SOₙ₁NR²R³, —NHNR²R³, —ONR²R³, —NHC(O)NHNR²R³, —NHC(O)NR²R³, —N(O)ₘ₁, —C(O)R⁵, —C(O)OR⁵, —C(O)NR²R³, —OR⁴, —NR²SO₂R⁴, —NR²C(O)R⁵, —NR²C(O)OR⁵, —NR²C(O)R⁵, —N₃, —NR²OR⁵, —OCX^{a}₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R², R³, R⁴, R⁵, R⁶, R⁷, R⁸, and R⁹ are independently hydrogen, halogen, —CX^{b}₃, —CHX^{b}₂, —CH₂X^{b}, —CN, —SO₂Cl, —SOₙ₂R⁶, —SOₙ₂NR⁷R⁸, —NHNH₂, —ONR⁷R⁸, —NHC(O)NHNH₂, —NHC(O)NR⁷R⁸, —N(O)ₘ₂, —NR⁷R⁸, —C(O)R⁹, —C(O)OR⁹, —C(O)NR⁷R⁸, —OR⁶, —NR⁷SO₂R⁶, —NR⁷C(O)R⁹, —NR⁷C(O)OR⁹, —NR⁷OR⁹, OCX^{b}₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ are optionally joined to form a substituted or unsubstituted heterocycloalkyl, or substituted or unsubstituted heteroaryl;
R⁷ and R⁸ are optionally joined to form a substituted or unsubstituted heterocycloalkyl or substituted or unsubstituted heteroaryl;
m1 and m2 are independently 1 or 2;
n1 and n2 are independently an integer from 0 to 2;
z1 is an integer from 0 to 4;
z2 is an integer from 0 to 5;
X^{a} and X^{b} are independently —Cl, —Br, —I, or —F.

2. The compound of claim 1, wherein R¹ is halogen, —NR²R³, —CX^{a}₃, —CN, —SO₂Cl, —SOₙ₁R⁴, —SOₙ₁NR²R³, —NHNR²R³, —ONR²R³, —NHC(O)NHNR²R³, —NHC(O)NR²R³, —N(O)ₘ₁, —C(O)R⁵, —C(O)—OR⁵, —C(O)NR²R³, —OR⁴, —NR²SO₂R⁴, —NR²C(O)R⁵, —NR²C(O)OR⁵, —NR²OR⁵, —OCX^{a}₃, —N₃, R^{1a}-substituted or unsubstituted alkyl, R^{1a}-substituted or unsubstituted heteroalkyl, R^{1a}-substituted or unsubstituted cycloalkyl, R^{1a}-substituted or unsubstituted heterocycloalkyl, R^{1a}-substituted or unsubstituted aryl, or R^{1a}-substituted or unsubstituted heteroaryl;
R^{1a} is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)NH₂, —NO₂, —C(O)H, —C(O)OH, —C(O)NH₂, —OH, —NHSO₂H, —NHC(O)H, —NHC(O)OH, —NHOH, —OCF₃, oxo, —N₃, R^{1b}-substituted or unsubstituted alkyl, R^{1b}-substituted or unsubstituted heteroalkyl, R^{1b}-substituted or unsubstituted cycloalkyl, R^{1b}-substituted or unsubstituted heterocycloalkyl, R^{1b}-substituted or unsubstituted aryl, or R^{1b}-substituted or unsubstituted heteroaryl; and
R^{1b} is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC═(O)H, —NHC(O)OH, —NHOH, —OCF₃, oxo, —N₃, unsubstituted alkyl, unsubstituted heteroalkyl, unsubstituted cycloalkyl, unsubstituted heterocycloalkyl, unsubstituted aryl, or unsubstituted heteroaryl.

3. The compound of claim 2, wherein R^{1b} is unsubsituted C₁-C₅ alkyl.

4. The compound of claim 1, wherein R², R³, R⁴, R⁵, R⁶, R⁷, R⁸ and R⁹ are independently selected from hydrogen, halogen, —CX^{b}₃, —CHX^{b}₂, —CH₂X^{b}, —CN, —SO₂Cl, —SOₙ₂R⁶, —SOₙ₂NR⁷R⁸, —NHNH₂, —ONR⁷R⁸, —NHC═(O)NHNH₂, —NHC(O)NR⁷R⁸, —N(O)ₘ₂, —NR⁷R⁸, —C(O)R⁹, —C(O)—OR⁹, —C(O)NR⁷R⁸, —OR⁶, —NR⁷SO₂R⁶, —NR⁷C(O)R⁹, —NR⁷C(O)OR⁹, —NR⁷OR⁹, —OCX^{b}₃, R¹⁹-substituted or unsubstituted alkyl, R¹⁰-substituted or unsubstituted heteroalkyl, R¹⁹-substituted or unsubstituted cycloalkyl, R¹⁰-substituted or unsubstituted heterocycloalkyl, R¹⁹-substituted or unsubstituted aryl, or R¹⁰-substituted and unsubstituted heteroaryl, wherein R² and R³ substituents are optionally joined to form R¹⁰-substituted or unsubstituted heterocycloalkyl, or R¹⁰-substituted or unsubstituted heteroaryl and R⁷ and R⁸ substituents are optionally joined to form R¹⁰-substituted or unsubstituted heterocycloalkyl, or R¹⁰-substituted or unsubstituted heteroaryl;
R¹⁰ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC═(O)H, —NHC(O)OH, —NHOH, —OCF₃, —N₃, oxo, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted and unsubstituted heteroaryl; and
R¹¹ is halogen, —NH₂, —CF₃, —CHF₂, —CH₂F, —CN, —SO₂Cl, —SH, —SO₂NH₂, —NHNH₂, —ONH₂, —NHC(O)NHNH₂, —NHC(O)N H₂, —NO₂, —C(O)H, —C(O)OH, —C(O)NH₂, —OH, —NHSO₂H, —NHC(O)H, —NHC(O)OH, —NHOH, —OCF₃, —N₃, oxo, unsubstituted alkyl, unsubstituted heteroalkyl, unsubstituted cycloalkyl, unsubstituted heterocycloalkyl, unsubstituted aryl, or unsubstituted heteroaryl.

5. The compound of claim 4, wherein R¹¹ is unsubstituted C₁-C₅ alkyl.

6. The compound of claim 4, wherein R² and R³ substituents are joined to form R¹⁰-substituted or unsubstituted heterocycloalkyl, or R¹⁰-substituted or unsubstituted heteroaryl.

7. The compound of claim 6, wherein R¹⁰ is unsubsituted C₁-C₅ alkyl.

8. The compound of claim 4, wherein R⁷ and R⁸ substituents are joined to form R¹⁰-substituted or unsubstituted heterocycloalkyl, or R¹⁰-substituted or unsubstituted heteroaryl.

9. The compound of claim 8, wherein R¹⁰ is unsubsituted C₁-C₅ alkyl

10. The compound of claim 3, wherein R^{1b} is methyl.

11. The compound of claim 10, wherein R^{1a} is R^{1b}-unsubstituted alkyl.

12. The compound of claim 11, wherein z1 is 2.

13. The compound of claim 11, wherein z1 is 1.

14. The compound of claim 11, wherein z2 is 2.

15. The compound of claim 11, wherein z2 is 1.

16. The compound of claim 1, wherein z1 is 0.

17. The compound of claim 1, wherein z2 is 0.

18. A pharmaceutical composition comprising a compound of claim 1 or a pharmaceutically acceptable salt thereof, and a pharmaceutically acceptable excipient.

19. A of treating cancer in a subject in need thereof, comprising administering to said subject an effective amount of a compound of claim 1, or a pharmaceutically acceptable salt thereof.
